(19) United States
(12) Patent Application Publication
(10) Pub. No.: US 2025/0381356 A1
(43) Pub. Date: Dec. 18, 2025

(54) SYSTEM AND METHOD FOR CHARACTERIZING AN INFLATABLE CUFF

(71) Applicant: AW Technologies ApS, Nørre Sundby (DK)

(72) Inventors: Casper Clement Borre, Hobro (DK); Dennis Schmidt Bennedbæk, Aalborg (DK); Adam Søholm Hansen, Aalborg SØ (DK); Andreas Achton Mortensen Hejslet, Aalborg (DK)

(73) Assignee: AW Technologies ApS, Nørre Sundby (DK)

(21) Appl. No.: 18/878,704

(22) PCT Filed: Jun. 26, 2023
(86) PCT No.: PCT/EP2023/067330

§ 371 Date: Dec. 24, 2024

(30) Foreign Application Priority Data
Jun. 27, 2022 (EP) 22181293.6

(51) Int. Cl. A61M 16/04

(52) U.S. Cl. CPC A61M 16/044; A61M 2205/07; A61M 2205/15; A61M 2205/3327; A61M 2205/3331; A61M 2205/50

(57) ABSTRACT

A system for characterizing an inflatable cuff, arranged around a tracheal tube for providing patient breathing support, includes at least one flow unit, such as a pump, in fluid connection with a cuff conduit connected to the cuff, to fill the cuff with gas or liquid. At least one pressure sensor measures pressure in the cuff. At least one flow sensor measures a flow of gas or liquid to and/or from the cuff. A processing unit estimates the volume or a change of volume in the cuff based on flow measurements. The processing unit is configured to: extract a pressure-volume relationship between the measured pressure and the estimated volume or a change of the volume in the cuff, and compare the pressure-volume relationship against a previously measured or a predetermined reference pressure-volume relationship to characterize the inflatable cuff. A method for characterizing such an inflatable cuff is also provided.

### CROSS-REFERENCE TO RELATED APPLICATIONS

This application is a U.S. National Phase of International Patent Application No. PCT/EP2023/067330, entitled “SYSTEM AND METHOD FOR CHARACTERIZING AN INFLATABLE CUFF”, filed on Jun. 26, 2023, which claims priority to European Patent Application No. 22181293.6, filed Jun. 27, 2022, the entire contents of each which are incorporated herein by reference in their entirety.

### FIELD OF THE INVENTION

The present disclosure relates to a system for characterizing an inflatable cuff arranged around a tracheal tube for providing breathing support to a patient. The disclosure further relates to method for characterizing an inflatable cuff arranged around a tracheal tube for providing breathing support to a patient.

### BACKGROUND

As known, ventilated patients, particularly those who are in intensive therapy, are intubated with endotracheal tubes or tracheal tubes. Many tubes have an inflatable cuff to seal the trachea against air leakage and aspiration of gastric contents, blood, secretions, and other fluids while allowing the patient to breathe through the tracheal tube.

The characteristics of such inflatable tube cuffs may deteriorate over time, which can cause leakage around the cuff. The presence of such leakage may decrease the efficiency of the ventilator and may also lead to bacterial aspiration to the lungs, potentially increasing the risk of pneumonia, which may prolong the time that the patient stays in the hospital. Patients that have pneumonia have a significantly increased length of hospital stay, a directly attributable increased mortality of and increased reintubation rate.

Several techniques are used to monitor and control the cuff pressure over time. For example, a pressure may be maintained above a certain minimum pressure or within a certain pressure range. However, monitoring the pressure of the cuff or maintaining a certain pressure in the cuff does not provide any detailed information about the status of the cuff, possible leakage around the cuff or other issues with the cuff or patient.

### SUMMARY

One object of the present disclosure is to provide a system for characterizing an inflatable cuff arranged around a tracheal tube for providing breathing support to a patient. A first aspect of the disclosure therefore relates to a system for characterizing an inflatable cuff arranged around a tracheal tube for providing breathing support to a patient, the system comprising:
at least one flow unit, such as a pump, in fluid connection with a cuff conduit connected to the inflatable cuff, wherein the at least one flow unit is adapted to fill the inflatable cuff with gas or liquid;
at least one pressure sensor configured to measure a pressure in the inflatable cuff;
at least one flow sensor for measuring a flow of gas or liquid to and/or from the inflatable cuff;
a processing unit for estimating the volume of gas or liquid or a change of the volume of gas or liquid in the inflatable cuff based on measurements of the flow of gas or liquid to and/or from the inflatable cuff, wherein the processing unit is configured to:
   extract a pressure-volume relationship between the measured pressure and the estimated volume of gas or liquid or a change of the volume of gas or liquid in the inflatable cuff, and
compare the pressure-volume relationship against a previously measured or a predetermined reference pressure-volume relationship to characterize the inflatable cuff.

By extracting a pressure-volume relationship between the measured pressure and the estimated volume of gas or liquid or a change of the volume of gas or liquid in the inflatable cuff, and comparing the pressure-volume relationship against a previously measured or a predetermined reference pressure-volume relationship, a change of the pressure-volume relationship over time may be obtained. The pressure-volume relationship may be, for example, a coefficient converting or translating at least one given volume to a corresponding pressure, or converting or translating at least one given pressure to a corresponding volume. The change of the pressure-volume relationship may be used to characterize the inflatable cuff non-invasively.

The characterization of the inflatable cuff may provide a measure of elasticity or of the inflatable cuff, but may also provide information about the structure around the inflatable cuff, usually the trachea. The pressure-volume relationship in a given configuration may be represented by a coefficient converting at least one given volume to a corresponding pressure, or converting at least one given pressure to a corresponding volume. A higher coefficient would normally mean that a higher pressure is required to obtain a given volume. As would be understood by a person skilled in the art, it would, equivalently, be possible to use a coefficient with the opposite meaning. The processing unit may then be used to interpret the change of the pressure-volume relationship. If the coefficient is less than a previously measured coefficient, it would mean that a lower pressure than in the first measurement is required to obtain a given volume. This may indicate a more elastic inflatable cuff. If the change of elasticity exceeds a predefined limit, the system may categorize the inflatable cuff as worn out. The predefined limit is typically not a limit for all inflatable cuffs in all patients be a limit for a specific inflatable cuff, possibly in a specific patient and possibly at a specific time. If the coefficient is higher than a previously measured coefficient, it would mean that a higher pressure than in the first measurement is required to obtain a given volume. Since the inflatable cuff does typically not get more difficult to fill with gas or liquid over time, the change to a higher coefficient may be interpreted as the structure around the inflatable cuff providing external pressure on the inflatable cuff. Hence, in one embodiment the system is further configured to identify a stiffer trachea of the patient as a result of an increase of the coefficient.

The disclosure further relates to a method for characterizing an inflatable cuff arranged around a tracheal tube for providing breathing support to a patient, the method comprising the steps of:
measuring a pressure in the inflatable cuff in the inflated configuration;
measuring a flow of gas to and/or from the inflatable cuff;
estimating the volume of gas or a change of the volume of gas or liquid in the inflatable cuff based on the measurements of the flow of gas to and/or from the inflatable cuff;
extracting a pressure-volume relationship between the measured pressure and the estimated volume of gas or a change of the volume of gas or liquid in the inflatable cuff, and
comparing the pressure-volume relationship against a previously measured or a predetermined reference pressure-volume relationship to characterize the inflatable cuff.

As would be understood by a person skilled in the art, the presently disclosed method for characterizing an inflatable cuff arranged around a tracheal tube for providing breathing support to a patient be performed using any embodiment of the presently disclosed system for characterizing an inflatable cuff arranged around a tracheal tube for providing breathing support to a patient, and vice versa.

The method may be implemented as a computer program having instructions, which, when executed by a computing device or computing system, cause the computing device or computing system to carry out the method for characterizing an inflatable cuff arranged around a tracheal tube for providing breathing support to a patient. A non-transitory storage medium may comprise a computer program product having instructions embodied thereon, wherein the computer program product, when executed by a computing device or system, causes the computing device or system to perform the method for characterizing an inflatable cuff arranged around a tracheal tube for providing breathing support to a patient.

### DESCRIPTION OF DRAWINGS

The invention will in the following be described with reference to the accompanying drawings. The drawings are examples of embodiments and not limiting to the presently disclosed system for characterizing an inflatable cuff arranged around a tracheal tube for providing breathing support to a patient.

FIG. 1 shows a schematic embodiment of the presently disclosed system for characterizing an inflatable cuff arranged around a tracheal tube.

FIG. 2 shows a further schematic embodiment of the presently disclosed system for characterizing an inflatable cuff arranged around a tracheal tube.

FIG. 3A-C show examples of processes for extracting a pressure-volume relationship between the measured pressure and the estimated volume of gas or liquid in the inflatable cuff.

FIG. 4A-C show examples of pressure-volume relationships between the measured pressure and the estimated volume of gas or liquid in the inflatable cuff.

FIG. 5A-C show an example of a sequence of inflating and deflating the inflatable cuff to establish a pressure-volume relationships.

FIG. 6A-C show an example of a sequence of deflating and inflating the inflatable cuff to establish a pressure-volume relationships.

FIG. 7 shows examples of pressure-volume relationships of the same inflatable cuff at different times.

### DETAILED DESCRIPTION

The present disclosure relates to a system for characterizing an inflatable cuff arranged around a tracheal tube for providing breathing support to a patient, the system comprising:
at least one flow unit, such as a pump, in fluid connection with a cuff conduit connected to the inflatable cuff, wherein the at least one flow unit is adapted to fill the inflatable cuff with gas or liquid;
at least one pressure sensor configured to measure a pressure in the inflatable cuff;
at least one flow sensor for measuring a flow of gas or liquid to and/or from the inflatable cuff;
a processing unit for estimating the volume of gas or liquid or a change of the volume of gas or liquid in the inflatable cuff based on measurements of the flow of gas or liquid to and/or from the inflatable cuff, wherein the processing unit is configured to:
   extract a pressure-volume relationship between the measured pressure and the estimated volume of gas or liquid or a change of the volume of gas or liquid in the inflatable cuff, and
   compare the pressure-volume relationship against a previously measured or a predetermined reference pressure-volume relationship to characterize the inflatable cuff.

By characterizing the inflatable cuff, in particular by analysing the pressure-volume relationship over time, conclusions can be made both regarding the inflatable cuff itself and the structure surrounding it. The characterization of the inflatable cuff may provide a measure of elasticity of the inflatable cuff and/or a measure of compliance. The system may be part of a ventilator or operate as an independent system.

The term ‘tracheal tube’ shall be construed broadly to comprise any suitable tube for maintaining a patient airway, including, but not limited to, endotracheal tubes and tracheostomy tubes. The inflatable cuff may accordingly be referred to as an inflatable endotracheal tube cuff or an inflatable tracheostomy tube cuff.

A ‘processing unit’ shall also be construed broadly to comprise any suitable means for processing and calculating data. The processing unit may be a local device, optionally integrated in a ventilator system, but may also be provided on a remote device, such as in a cloud computing device.

A flow sensor can be used to measure a flow rate of a gas or liquid. All types of flow sensor may be used to a flow of gas or liquid to and/or from the inflatable cuff according to the present disclosure. Based on the flow of gas or liquid over some time, a volume of gas or liquid or a change of the volume of gas or liquid in the inflatable cuff can be estimated. The presently disclosed system for characterizing an inflatable cuff is not limited to a specific flow sensor.

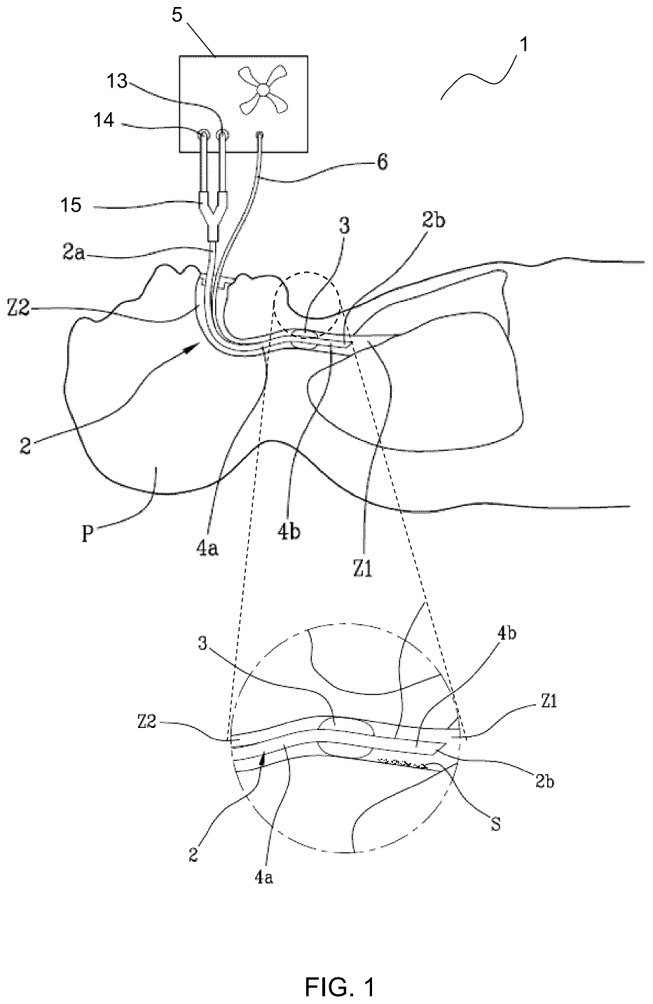

FIG. 1 shows a schematic embodiment of the general concept of intubation of a patient P with a tracheal tube 2 and an inflatable cuff 3. A system 1 for characterizing an inflatable cuff 3 arranged around a tracheal tube 3 for providing breathing support to a patient P is disclosed. As mentioned, the system 1 is connectable to or integrated in a ventilator for ventilation of the patient P. The tracheal tube 2 has a sidewall 2c and extends between a first end 2a and a second end 2b. The tracheal tube 2 is preferably made of PVC or the like, but may be made of any suitable material. The tracheal tube 2 has an associated inflatable cuff 3. The inflatable cuff 3 may be configured to seal the trachea of a patient P in order to isolate the second end 2b from the external environment. The inflatable cuff 3 may be an annular element externally fitted to the sidewall 2c of the tracheal tube 2. The inflatable cuff 3 divides the tracheal tube 2 into a first portion 4a and a second portion 4b. When positioned in the trachea, the inflatable cuff 3 also divides the trachea of the patient into an isolated zone Z1 and a non-sterile zone Z2.

The inflatable cuff 3 may have a substantially annular shape (donut-shaped). As would be recognized by a person skilled in the art, the inflatable cuff 3 may have any other suitable shape. Preferably, the inflatable cuff 3 is placed in fluid connection with at least a flow unit 5 through a conduit 6. The inflatable cuff 3 may have a chamber bound by an outer membrane. The outer membrane may be made of, for example, PVC less than 100 microns thick, or polyurethane having a thickness of less than 50 microns.

A flow unit 5 may also be connected to the tracheal tube 2. This may be a different flow unit than the one used for the inflatable cuff 3. It may be configured to generate a positive pressure and pause pressure and it may be are connected to the tracheal tube 2 through at least one connection mouth 13; 14 having an inlet mouth 13 and an outlet mouth 14, both being in fluid connection with the tracheal tube 2 through a two-input one-output connector 15.

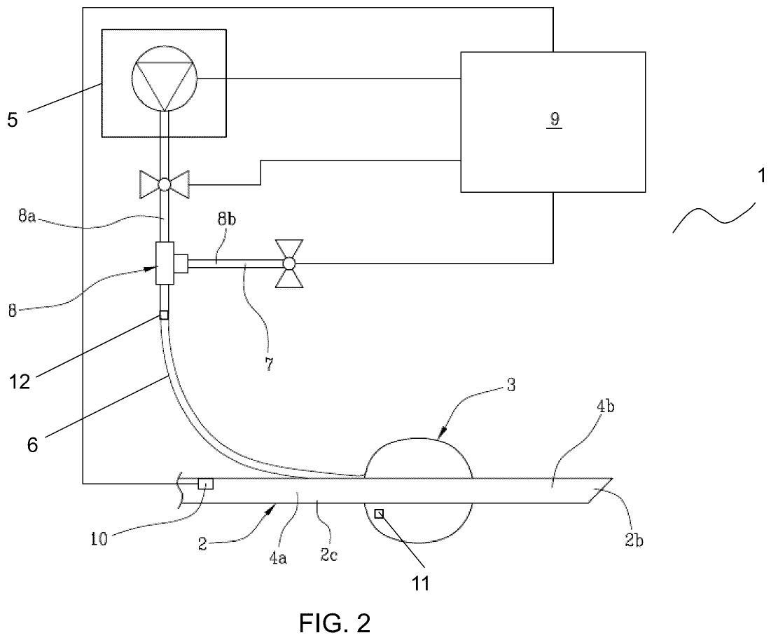

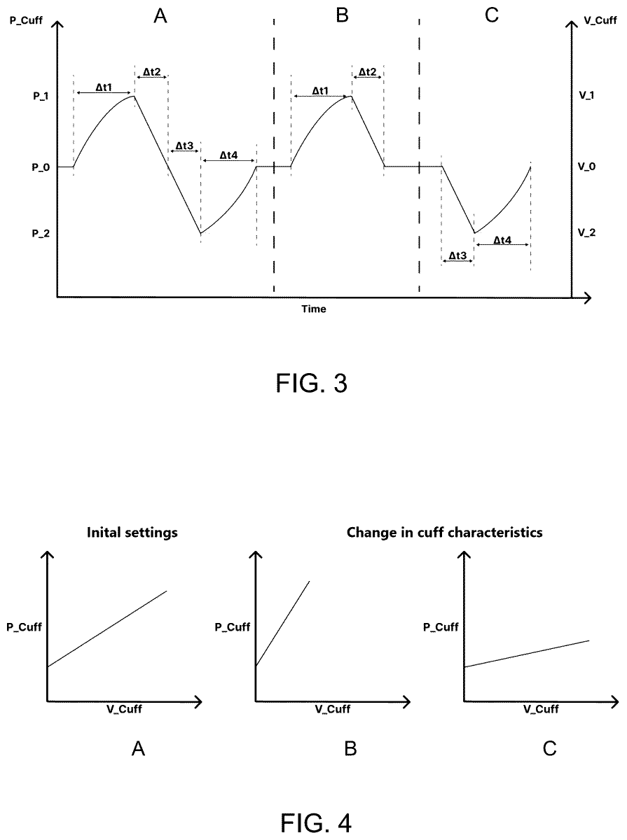

FIG. 2 shows a further schematic embodiment of the presently disclosed system 1 for characterizing an inflatable cuff 3 arranged around a tracheal tube 2. In FIG. 3 a processing unit or control unit 9 controls the flow unit 5. The processing unit 9 may also estimate the volume of gas or liquid or a change of the volume of gas or liquid in the inflatable cuff based on measurements of the flow of gas or liquid to and/or from the inflatable cuff 3 based on a flow sensor 12. The flow sensor 12 may be placed at any suitable position. A pressure sensor 11 is configured to measure a pressure in the inflatable cuff 3. The pressure sensor 11 may be placed at any suitable position. The system 1 in the example of fig. has a venting conduit 7, which can be used for a parallel flow. The system 1 of. FIG. 2 further comprises a fork 8 placed along the conduit 6.

According to an aspect of the present disclosure, the system and/or method comprises a step of inflating or deflating, or a combination of both, said inflatable cuff, either controlled by flow or pressure, in order to estimate the endotracheal tube cuff or tracheal tube cuff characteristics in an intubated patient.

Preferably, the step of generating a succession of inflating or deflating or a combination of both, said inflatable cuff, either controlled by flow or pressure, comprises steps of generating a cyclic succession of either an overpressure inside the inflatable cuff followed by a normal pressure setting inside the inflatable cuff, or an underpressure inside the inflatable cuff followed by a normal pressure setting inside the inflatable cuff, or a combination of both, in order to measure a pressure/volume correlation inside the inflatable cuff.

The flow unit may be switchable between an inflation configuration, in which it inflates and/or maintains inflated the inflatable cuff, and a deflation configuration, wherein it deflates and/or maintains inflated the inflatable cuff, or a combination of inflation/deflation configuration.

In accordance with the presently disclosed system and method, the flow unit may be switched in a cyclical succession of first inflation configuration, deflation configuration or a combination of both, either manually or automatically.

The control module, which may comprise a processing unit, may be configured to control signals to the flow unit in order to activate or deactivate inflation or deflation. This may involve opening and closing of valves.

In one embodiment the system is configured to determine a degradation of the inflatable cuff based on the characterization of the inflatable cuff. A degradation of an inflatable cuff may be show as the inflatable cuff loses is resilience i.e. its ability to endure tribulation without cracking. A degradation may also be seen as a more elastic inflatable cuff. The processing unit is configured to compute or extract a measure of elasticity of the inflatable cuff based on the pressure-volume relationship.

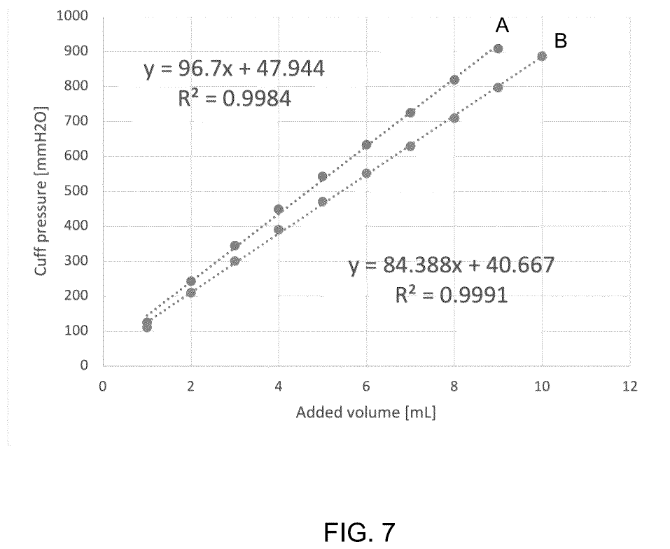

The pressure-volume relationship may describe a relationship between one or more given volume(s) and one or more corresponding pressure(s). The pressure-volume relationship describes a relationship between one or more given pressure(s) and one or more corresponding volumes(s). This can be illustrated, for example, by FIG. 7, in which the volumes and pressures for two inflatable cuffs (or the same inflatable cuff at different points in time) A and B are shown. The pressure-volume relationship may be represented by a pressure-volume curve including measurements of a plurality of pressure levels and a plurality of volumes extracted from a plurality of measurements of flow of gas to and/or from the inflatable cuff. The pressure is provided on a first axis, such as a Y-axis, and the volume is provided on a second axis, such as an X-axis, of the curve. The pressure-volume relationship may be represented by a coefficient or formula converting at least one given volume to a corresponding pressure, or converting at least one given pressure to a corresponding volume. If it is a single pressure and volume it can be a single coefficient. If there are a number of measurements, the relationship can be expressed as an equation. A a higher coefficient may indicate that a higher pressure is required to obtain the given volume, and a lower coefficient may indicate that a lower pressure is required to obtain the given volume, or a higher coefficient may indicate that a smaller volume is required to obtain the given pressure, and a lower coefficient may indicate that a greater volume is required to obtain the given pressure. In the example of FIG. 7 the inflatable cuff of scenario A a higher pressure is required to obtain the given volume compared to scenario B. Scenarios A and B could be for the same inflatable cuff. If B occurs later than A, it may be seen as B has become more elastic. If the pressure is provided on the Y-axis and the volume is provided on the X-axis, as in the example of FIG. 7, a steeper curve may indicate a less elastic inflatable cuff and/or a higher coefficient, and a flatter curve indicates a more elastic inflatable cuff and/or a lower coefficient. In one embodiment, the processing unit is configured to categorize the inflatable cuff as worn out based on a change of the pressure-volume relationship over time. If the coefficient is less than a previously measured coefficient, the processing unit may be configured to categorize the inflatable cuff as worn out. Possibly the coefficient needs to be more than a predefined limit lower than previously measured coefficient in order for the inflatable cuff to be categorized as worn out.

The processing unit may additionally, or alternatively, be configured to identify a change in the structure around the cuff as a change in the pressure-volume relationship. The system may accordingly be configured to characterize a structure around the inflatable cuff based on the characterization of the inflatable cuff. The processing unit may be configured to identify a stiffer trachea of the patient as a result of an increase of the previously described coefficient, and/or configured to identify a softer trachea of the patient as a result of a decrease of the coefficient. In the example of FIG. 7, if scenario B represents a first round of measurements and scenario A represents a subsequent second round of measurements, the higher coefficient of A may indicate that the trachea of the patient is stiffer.

In order to characterize the inflatable cuff, the system may be configured to collect the needed measurements. In one embodiment, the system may be configured to gradually fill the inflatable cuff with gas, or gradually empty the inflatable cuff, while the pressure sensor and the flow sensor, continuously, or at discrete points in time, measures the pressure in the inflatable cuff and the flow of gas to and/or from the inflatable cuff to generate a pressure-volume curve.

FIG. 3A-C show examples of processes for extracting a pressure-volume relationship between the measured pressure and the estimated volume of gas or liquid in the inflatable cuff.

In scenario A, the inflatable cuff is first inflated from a first pressure P_0 or first volume V_0 to a second pressure P_1 or a second volume V_1 in a second time interval Δt1. Preferably, both the pressure and the volume are measured, continuously, or at discrete points in time. The second pressure P_1 or second volume V_1 is then decreased to a third pressure P_2 or a third volume V_2 in a second and third time interval Δt2 and Δt3. The third pressure P_2 or third volume V_2 is then increased back to the first pressure P_0 or the first volume V_0 in a fourth time interval Δt4.

In scenario B, the inflatable cuff is first inflated from a first pressure P_0 or first volume V_0 to a second pressure P_1 or a second volume V_1 in a second time interval Δt1. The second pressure P_1 or second volume V_1 is then decreased to the first pressure P_0 or the first volume V_0 in a second time interval Δt2.

In scenario C, the inflatable cuff is first deflated from a first pressure P_0 or first volume V_O to a third pressure P_2 or a third volume V_2 in a third time interval Δt3. The third pressure P_2 or third volume V_2 is then increased to the first pressure P_0 or the first volume V_0 in a fourth time interval Δt4.

Preferably, the time intervals are at least 0.01 seconds.

Preferably, the second pressure is at least 1 cmH2O higher than the first pressure. Preferably, the third pressure is at least 1 cmH2O lower than the first pressure.

Preferably, the second volume is at least 0.1 mL greater than the first volume. Preferably, the third volume is at least 0.1 mL smaller than the first volume.

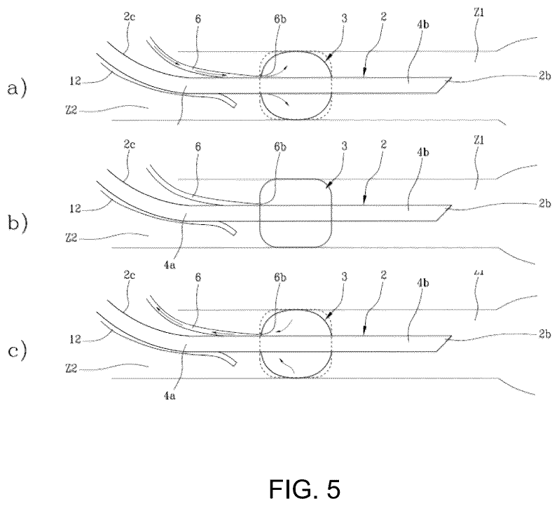

FIG. 5A-C show an example of a sequence of inflating and deflating the inflatable cuff to establish a pressure-volume relationships. The inflatable cuff starts in an initial state shown in FIG. 5A. The pressure is then increased to a second state shown in FIG. 5B. The pressure is then decreased to a third state shown in FIG. 5C.

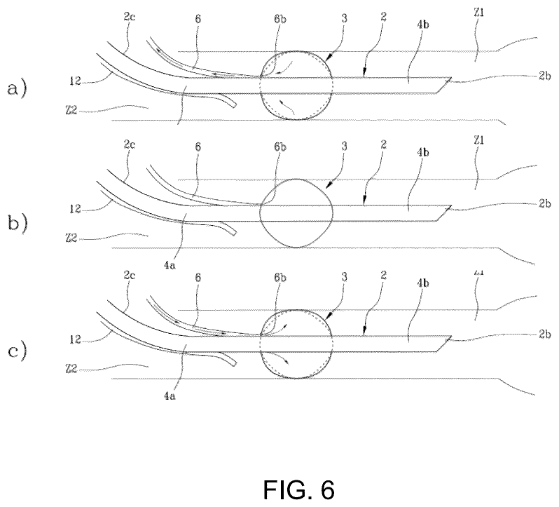

FIG. 6A-C show an example of a sequence of deflating and inflating the inflatable cuff to establish a pressure-volume relationships The inflatable cuff starts in an initial state shown in FIG. 6A. The pressure is then decreased to a second state shown in FIG. 6B. The pressure is then increased to a third state shown in FIG. 6C.

Advantageously, in this way it is possible to characterize the tracheal tube in an intubated patient by correlating the measured pressure and volume of the inflatable as shown in FIG. 4. FIG. 4A-C show examples of pressure-volume relationships between the measured pressure and the estimated volume of gas or liquid in the inflatable cuff. FIG. 4A may be seen as an initial pressure-volume relationship between the pressure and volume of gas or liquid in the inflatable cuff. FIGS. 4B and C may then be subsequent measurements of pressure-volume relationship between the pressure and volume of gas or liquid in the inflatable cuff. FIG. 4B may in this regard represent a stiffer trachea, whereas FIG. 4C may represent a more elastic inflatable cuff.

The inflation volume of the inflatable cuff is to be determined according to type of patient and is therefore proportional to the size of the trachea and correlated to a target pressure to be generated in the inflatable cuff. However, the pressure inside the inflatable cuff may be determined independently of the volume. Indicatively, the pressure P_0 within the inflatable cuff, in order to prevent ischemia, is, preferably, between 20 and 30 cmH2O.

The disclosure further relates to a method for characterizing an inflatable cuff arranged around a tracheal tube for providing breathing support to a patient, the method comprising the steps of:
measuring a pressure in the inflatable cuff in the inflated configuration;
measuring a flow of gas to and/or from the inflatable cuff;
estimating the volume of gas in the inflatable cuff based on the measurements of the flow of gas to and/or from the inflatable cuff;
extracting a pressure-volume relationship between the measured pressure and the estimated volume of gas in the inflatable cuff, and
comparing the pressure-volume relationship against a previously measured or a predetermined reference pressure-volume relationship to characterize the inflatable cuff.

The method may comprise the steps of:
gradually filling the inflatable cuff with gas, and/or gradually emptying the inflatable cuff;
continuously, or at discrete points in time, measuring the pressure in the inflatable cuff and the flow of gas to and/or from the inflatable cuff to generate a pressure-volume curve.

### Further Details

1. A system for characterizing an inflatable cuff arranged around a tracheal tube for providing breathing support to a patient, the system comprising:
   at least one flow unit, such as a pump, in fluid connection with a cuff conduit connected to the inflatable cuff, wherein the at least one flow unit is adapted to fill the inflatable cuff with gas or liquid;
   at least one pressure sensor configured to measure a pressure in the inflatable cuff;
   at least one flow sensor for measuring a flow of gas or liquid to and/or from the inflatable cuff;
   a processing unit for estimating the volume of gas or liquid or a change of the volume of gas or liquid in the inflatable cuff based on measurements of the flow of gas or liquid to and/or from the inflatable cuff, wherein the processing unit is configured to:
      extract a pressure-volume relationship between the measured pressure and the estimated volume of gas or liquid or change of the volume of gas or liquid in the inflatable cuff, and
      compare the pressure-volume relationship against a previously measured or a predetermined reference pressure-volume relationship to characterize the inflatable cuff.
2. The system for characterizing an inflatable cuff according to item 1, wherein the system is configured to determine a degradation of the inflatable cuff based on the characterization of the inflatable cuff.
3. The system for characterizing an inflatable cuff according to any one of the preceding items, wherein the system is further configured to characterize a structure around the inflatable cuff based on the characterization of the inflatable cuff.
4. The system for characterizing an inflatable cuff according to any one of the preceding items, wherein the pressure-volume relationship describes a relationship between one or more given volume(s) and one or more corresponding pressure(s), or wherein the pressure-volume relationship describes a relationship between one or more given pressure(s) and one or more corresponding volumes(s).
5. The system for characterizing an inflatable cuff according to any one of the preceding items, wherein the pressure-volume relationship is represented by a coefficient converting at least one given volume to a corresponding pressure, or converting at least one given pressure to a corresponding volume.
6. The system for characterizing an inflatable cuff according to item 5, wherein a higher coefficient indicates that a higher pressure is required to obtain the given volume, and a lower coefficient indicates that a lower pressure is required to obtain the given volume, or a higher coefficient indicates that a smaller volume is required to obtain the given pressure, and a lower coefficient indicates that a greater volume is required to obtain the given pressure.
7. The system for characterizing an inflatable cuff according to any one of items 4 to 6, wherein the processing unit is configured to categorize the inflatable cuff as worn out based on a change of the pressure-volume relationship over time.
8. The system for characterizing an inflatable cuff according to item 7, wherein if the coefficient is less than a previously measured coefficient, the processing unit is configured to categorize the inflatable cuff as worn out.
9. The system for characterizing an inflatable cuff according to any one of the preceding items, wherein the pressure-volume relationship is represented by a pressure-volume curve including measurements of a plurality of pressure levels and a plurality of volumes extracted from a plurality of measurements of flow of gas to and/or from the inflatable cuff.
10. The system for characterizing an inflatable cuff according to item 9, wherein the pressure is provided on a first axis, such as a Y-axis, and the volume is provided on a second axis, such as an X-axis, of the curve.
11. The system for characterizing an inflatable cuff according to any one of items 9 to 10, wherein a steeper curve indicates a less elastic inflatable cuff and/or a higher coefficient, and a flatter curve indicates a more elastic inflatable cuff and/or a lower coefficient.
12. The system for characterizing an inflatable cuff according to any one of the preceding items, wherein the at least one flow unit is configured to gradually fill the inflatable cuff with gas, or gradually empty the inflatable cuff, while the pressure sensor and the flow sensor, continuously, or at discrete points in time, measures the pressure in the inflatable cuff and the flow of gas to and/or from the inflatable cuff to generate a pressure-volume curve.
13. The system for characterizing an inflatable cuff according to any one of the preceding items, wherein the processing unit is configured to compute or extract a measure of elasticity of the inflatable cuff based on the pressure-volume relationship.
14. The system for characterizing an inflatable cuff according to any one of the preceding items, wherein the processing unit is further configured to identify a change in the structure around the cuff as a change in the pressure-volume relationship.
15. The system for characterizing an inflatable cuff according to any one of items 5 to 6, wherein the processing unit is further configured to identify a stiffer trachea and/or a smaller inner diameter of the trachea of the patient as a result of an increase of the coefficient, and/or wherein the processing unit is further configured to identify a softer trachea and/or a larger inner diameter of the trachea of the patient based on a change of the pressure-volume relationship over time
16. The system for characterizing an inflatable cuff according to any one of items 5 to 6, wherein the processing unit is further configured to identify a stiffer trachea and/or a smaller inner diameter of the trachea of the patient as a result of an increase of the coefficient, and/or wherein the processing unit is further configured to identify a softer trachea and/or a larger inner diameter of the trachea of the patient as a result of a decrease of the coefficient.
17. The system for characterizing an inflatable cuff according to any one of the preceding items, wherein the inflatable cuff is an inflatable endotracheal tube cuff or inflatable tracheostomy tube cuff.
18. A method for characterizing an inflatable cuff arranged around a tracheal tube for providing breathing support to a patient, the method comprising the steps of:
   measuring a pressure in the inflatable cuff in the inflated configuration;
   measuring a flow of gas to and/or from the inflatable cuff;
   estimating the volume of gas or a change of the volume of gas or liquid in the inflatable cuff based on the measurements of the flow of gas to and/or from the inflatable cuff;
   extracting a pressure-volume relationship between the measured pressure and the estimated volume of gas in the inflatable cuff, and
   comparing the pressure-volume relationship against a previously measured or a predetermined reference pressure-volume relationship to characterize the inflatable cuff.
19. The method for characterizing an inflatable cuff arranged around a tracheal tube for providing breathing support to a patient according to item 18, comprising the steps of:
   gradually filling the inflatable cuff with gas, and/or gradually emptying the inflatable cuff;
   continuously, or at discrete points in time, measuring the pressure in the inflatable cuff and the flow of gas to and/or from the inflatable cuff to generate a pressure-volume curve.
20. The method according to any one of items 18 to 19 carried out on the system for characterizing an inflatable endotracheal tube cuff according to any one of items 1 to 17.
21. A computer program having instructions which, when executed by a computing device or computing system, cause the computing device or computing system to carry out the method for characterizing an inflatable cuff arranged around a tracheal tube for providing breathing support to a patient according to any one of items 18 to 20.

1. A system for characterizing an inflatable cuff arranged around a tracheal tube for providing breathing support to a patient, the system comprising:
at least one flow unit, such as a pump, in fluid connection with a cuff conduit connected to the inflatable cuff, wherein the at least one flow unit is adapted to fill the inflatable cuff with gas or liquid;
at least one pressure sensor configured to measure a pressure in the inflatable cuff;
at least one flow sensor for measuring a flow of gas or liquid to and/or from the inflatable cuff;
a processing unit for estimating the volume of gas or liquid or a change of the volume of gas or liquid in the inflatable cuff based on measurements of the flow of gas or liquid to and/or from the inflatable cuff, wherein the processing unit is configured to:
extract a pressure-volume relationship between the measured pressure and the estimated volume of gas or liquid or change of the volume of gas or liquid in the inflatable cuff, and
compare the pressure-volume relationship against a previously measured or a predetermined reference pressure-volume relationship to characterize the inflatable cuff.

2. The system for characterizing an inflatable cuff according to claim 1, wherein the system is configured to determine a degradation of the inflatable cuff based on the characterization of the inflatable cuff.

3. The system for characterizing an inflatable cuff according to claim 1, wherein the system is further configured to characterize a structure around the inflatable cuff based on the characterization of the inflatable cuff.

4. The system for characterizing an inflatable cuff according to claim 1, wherein the pressure-volume relationship describes a relationship between one or more given volume(s) and one or more corresponding pressure(s), or wherein the pressure-volume relationship describes a relationship between one or more given pressure(s) and one or more corresponding volumes(s).

5. The system for characterizing an inflatable cuff according to claim 1, wherein the pressure-volume relationship is represented by a coefficient converting at least one given volume to a corresponding pressure, or converting at least one given pressure to a corresponding volume.

6. The system for characterizing an inflatable cuff according to claim 5, wherein a higher coefficient indicates that a higher pressure is required to obtain the given volume, and a lower coefficient indicates that a lower pressure is required to obtain the given volume, or a higher coefficient indicates that a smaller volume is required to obtain the given pressure, and a lower coefficient indicates that a greater volume is required to obtain the given pressure.

7. The system for characterizing an inflatable cuff according to claim 4, wherein the processing unit is configured to categorize the inflatable cuff as worn out based on a change of the pressure-volume relationship over time, preferably, wherein, if the coefficient is less than a previously measured coefficient, the processing unit is configured to categorize the inflatable cuff as worn out.

8. The system for characterizing an inflatable cuff according to claim 1, wherein the pressure-volume relationship is represented by a pressure-volume curve including measurements of a plurality of pressure levels and a plurality of volumes extracted from a plurality of measurements of flow of gas to and/or from the inflatable cuff.

9. The system for characterizing an inflatable cuff according to claim 1, wherein the at least one flow unit is configured to gradually fill the inflatable cuff with gas, or gradually empty the inflatable cuff, while the pressure sensor and the flow sensor, continuously, or at discrete points in time, measures the pressure in the inflatable cuff and the flow of gas to and/or from the inflatable cuff to generate a pressure-volume curve.

10. The system for characterizing an inflatable cuff according to claim 1, wherein the processing unit is configured to compute or extract a measure of elasticity of the inflatable cuff based on the pressure-volume relationship.

11. The system for characterizing an inflatable cuff according to claim 1, wherein the processing unit is further configured to identify a change in the structure around the cuff as a change in the pressure-volume relationship.

12. The system for characterizing an inflatable cuff according to claim 5, wherein the processing unit is further configured to identify a stiffer trachea and/or a smaller inner diameter of the trachea of the patient as a result of an increase of the coefficient, and/or wherein the processing unit is further configured to identify a softer trachea and/or a larger inner diameter of the trachea of the patient based on a change of the pressure-volume relationship over time.

13. The system for characterizing an inflatable cuff according to claim 5, wherein the processing unit is further configured to identify a stiffer trachea and/or a smaller inner diameter of the trachea of the patient as a result of an increase of the coefficient, and/or wherein the processing unit is further configured to identify a softer trachea and/or a larger inner diameter of the trachea of the patient as a result of a decrease of the coefficient.

14. A method for characterizing an inflatable cuff arranged around a tracheal tube for providing breathing support to a patient, the method comprising the steps of:
measuring a pressure in the inflatable cuff in the inflated configuration;
measuring a flow of gas to and/or from the inflatable cuff;
estimating the volume of gas or a change of the volume of gas or liquid in the inflatable cuff based on the measurements of the flow of gas to and/or from the inflatable cuff;
extracting a pressure-volume relationship between the measured pressure and the estimated volume of gas in the inflatable cuff, and
comparing the pressure-volume relationship against a previously measured or a predetermined reference pressure-volume relationship to characterize the inflatable cuff.

15. A non-transitory storage medium comprising a computer program product having instructions which, when executed by a computing device or computing system, cause the computing device or computing system to carry out the method for characterizing an inflatable cuff arranged around a tracheal tube for providing breathing support to a patient according to claim 14.
